(19) United States
(12) Patent Application Publication
(10) Pub. No.: US 2006/0247745 A1
(43) Pub. Date: Nov. 2, 2006

(54) Garment for the prevention or treatment of hypothermia and methods of treatment

(76) Inventor: Chris Thompson, Beaufort, SC (US)

Correspondence Address: DAVID R PRESTON & ASSOCIATES APC, 5850 OBERLIN DRIVE, SUITE 300, SAN DIEGO, CA, 92121, US

(21) Appl. No.: 11/411,624

(22) Filed: Apr. 26, 2006

Related U.S. Application Data
(60) Provisional application No. 60/675,933, filed on Apr. 28, 2005.

(51) Int. Cl. A61F 7/00

(52) U.S. Cl. 607/108; 607/109; 607/114

(57) ABSTRACT

The present invention includes a hypothermia protection garment including a reversibly closable upper body portion for substantially covering the upper body of an individual, the upper body portion including a torso portion capable of substantially covering the torso of the individual, two sleeve portions capable of substantially covering the arms and optionally the hands of the individual and a hood portion capable of substantially covering the head of an individual, the hood portion includes a face aperture or opening for exposing a portion of the face of the individual. The upper body portion has a heat reflective inner surface and a heat positioning structure for positioning a dry active heating means. The garment may also include one or more medical access apertures capable of exposing a region of the individual for the application of a medical device or a medical procedure without substantial removal of the garment.

### CROSS REFERENCE TO RELATED APPLICATIONS

This application claims benefit of priority to U.S. patent application Ser. No. 60/675,933, filed on Apr. 28, 2005, which is herein incorporated by reference in its entirety.

### TECHNICAL FIELD

The present invention relates generally to temperature protection suits and more specifically to a garment for the prevention or treatment of hypothermia having a heat reflective inner surface and a heat positioning structure for positioning a dry active heating means or having an integrated dry active heating means.

### BACKGROUND

Hypothermia is a medical condition in which the victim's core body temperature drops below 35 degrees Celsius or 95 degrees Fahrenheit. Once the body temperature falls below 32 degrees Celsius or 90 degrees Fahrenheit the condition can become critical and eventually fatal. Body temperatures below 27 degrees Celsius or 80 degrees Fahrenheit are almost always uniformly fatal, though individuals having body temperatures as low as about 14 degrees Celcius have been known to survive. There are three types of hypothermia, acute, subacute and chronic. Acute hypothermia is the most dangerous. Acute hypothermia is swift drop of body temperature, often in a matter of seconds, such as when a victim falls through an ice-covered lake. Subacute hypothermia occurs on a scale of hours, most commonly by remaining in a cold environment for an extended period of time. Chronic hypothermia is typically caused by an underlying disease.

There are a variety of devices for the treatment or prevention of hypothermia. These range from passive temperature protection suits to warming blankets to isolation chambers. However, each technology has drawbacks or inefficiencies, some but not all of which are provided below.

U.S. patent Ser. No. 3,849,802 discloses a temperature protection suit. The suit has an upper body portion including arm portions and a hood portion. The upper body, arm and hood portions are made of Tyvek® fabric having one side coated with a very thin layer of shiny, heat reflective metal and a co-operating reversible releasable sealing fastening means along the split of the upper body and hood portions. While the disclosed suit is able to reflect heat back to the body, the suit is limited to passive heating and does not provide a means for actively heating the body or a means for retaining a dry active heating means. Therefore the disclosed suit relies solely on the individual's body temperature to generate heat. In addition, the suit does not include a medical access aperture for an emergency medical technician to monitor the progress of the patient. Therefore, it may be difficult to monitor a patient's heart rate or blood pressure or to inject medications or intravenous (IV) fluids without substantial removal of the protection suit.

U.S. patent Ser. No. 4,137,586 discloses a survival suit including a survival jacket and a crotch flap connected to the jacket for preventing the jacket from riding upward on the body. The survival jacket includes an outer floatation jacket and an inner jacket having thermal insulation properties and elastic properties. While the disclosed jacket is able to insulate the body from the outer elements, the jacket does not provide a means for actively heating the body or a means for positioning a dry active heating means. Therefore the disclosed suit relies solely on the individual's body temperature to generate heat. In addition, the jacket does not include a medical access aperture for an emergency medical technician to monitor the progress of the patient. Therefore, it may be difficult to monitor a patient's heart rate or blood pressure or to inject medications or intravenous (IV) fluids without substantial removal of the jacket.

U.S. patent Ser. No. 5,292,347 discloses a method of quickly raising a patient's body temperature including enclosing a portion of the patient's body such that the body is isolated from the external environment, generating a heated vapor, inputting the heated vapor to the enclosure such that the vapor condenses on the patient's body and monitoring the patient's temperature. Thus, the disclosed method warms the patient by creating a warm, damp, vaporous environment. However, the disclosed method utilizes moisture to warm the body and therefore requires the generation and delivery of a heated water vapor. This can be difficult to generate and deliver in the field for a survivalist, camper, ski patrol and the like. In addition, the device does not include a medical access aperture allowing an emergency medical technician to monitor the patient during treatment.

U.S. patent Ser. No. 5,360,439 discloses a warming blanket having multiple inlets with a force convection system. Although the disclosed warming blanket is capable of providing warm air to the patient, the device requires the generation and delivery of forced warm air. Moreover, the blanket does not include a medical access aperture for an emergency medical technician to monitor the progress of the patient. Thus, it may be difficult to monitor a patient's heart rate or blood pressure or to inject medications or intravenous (IV) fluids without substantial removal of the blanket.

U.S. patent application Ser. No. 5,383,918 discloses a hypothermia reducing body enclosure including a body suit capable of being filled with warm water. While warm water applied to a patient may warm the patient, the suit requires the generation and delivery of warm water. In addition, the disclosed body enclosure does not include an efficient means for an emergency medical technician to monitor the progress of or treat the victim. Thus, it may be difficult to monitor a patient's heart rate or blood pressure or to inject medications or intravenous (IV) fluids without substantial removal of the protection suit.

Although the above technologies provide some help in treating a victim suffering from or at risk of developing hypothermia, there remains a need to further develop devices with applications in this field.

### SUMMARY

The present invention address deficiencies in the field of hypothermia prevention and treatment and provides related benefits. More specifically, the present invention includes a hypothermia protection garment for the treatment or prevention of hypothermia. The garment warms the victim suffering from or at risk of developing hypothermia by passively reflecting body heat released by the individual back towards the body and is adapted to actively warm the victim by positioning a dry active heating means toward desired regions of the body. The active heating of the individual does not require the generation of forced air or heated and forced fluid. This combination results in a portable device capable of efficiently warming an individual in need of treatment.

One aspect of the present invention includes a hypothermia protection garment including a reversibly closable upper body portion for substantially covering the upper body of an individual, the upper body portion including a torso portion capable of substantially covering the torso of the individual, two sleeve portions capable of substantially covering the arms and optionally the hands of the individual and a hood portion capable of substantially covering the head of an individual, the hood portion includes a face aperture for exposing a portion of the face of the individual. The upper body portion has a heat reflective inner surface and a heat positioning structure for positioning a dry active heating means. The garment may also include one or more medical access apertures capable of exposing a region of the individual for the application of a medical device or a medical procedure without substantial removal of the garment. The medical access aperture may be reversibly opened and closed. The garment may also include a permanently attached or reversibly attachable hood portion for a head, sleeve portions for arms, glove portions for hands, a lower portion including leg portions for legs and sock portions for feet. The garment may also include an integrated dry heating means or reversibly positionable dry heating means.

In another aspect of the present invention a method of treating an individual suffering from or at risk of developing hypothermia is disclosed including placing the individual in need of such treatment or prevention in the hypothermia protection garment, activating a dry heating means and positioning the dry heating means in a desired location within the garment.

In yet another aspect of the present invention a method of treating an individual suffering from or at risk of developing hypothermia is disclosed including placing the individual in need of such treatment or prevention in the hypothermia protection garment having an integrated dry heating means and activating an integrated dry heating means.

### BRIEF DESCRIPTION OF THE DRAWINGS

Various non-limiting embodiments of the present invention are depicted in the following drawings to assist the reader in making and using the present inventions:

FIG. 1 depicts a front view of one embodiment of a hypothermia protection garment 10 of the present invention in its closed state and positioned about the torso of an individual. The upper body portion includes a torso portion 12, two sleeve portions 14, a hood potion 16 including a face aperture 18 for exposure of a portion of the face, a crotch flap 18, medical access apertures 22 positioned across the chest and arms 22a, along the chest 22b and along the abdominal region or midsection 22c.

FIG. 2 depicts a front view of the hypothermia protection garment 10 shown in FIG. 1 in its open position. The reflective inner surface 24 is demonstrated as well as the crotch flap 18 in its opened position.

FIG. 3 depicts a front view of the hypothermia protection garment 10 shown in FIG. 1 demonstrating medical access apertures 22 that may be reversibly closed. Medical access apertures 22 along the sleeves 22a and along the abdominal region or midsection 22c are shown in their open position, which allows access to the individual without substantial removal of the garment 10.

FIG. 4 depicts a rear view of the hypothermia protection garment 10 shown in FIG. 1 including a reversibly closable rear medical access aperture 22d along the back.

FIG. 5 depicts a front view of the hypothermia protection garment 10 shown in FIG. 1 in an open position. The heat reflective inner surface 24 is shown as well as heat positioning structures capable of housing or positioning a dry active heating means 26. The heat positioning structures are positioned at a neck region 26a, an armpit region 26b and a lower waist or hip region 26c.

### DETAILED DESCRIPTION OF THE INVENTION

Definitions

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there is a plurality of definitions for a term herein, those in this section prevail unless stated otherwise.

The term “torso portion” as used herein refers to the portion of a garment that covers or is adorned about the torso of an individual. The “torso portion” does not substantially cover the legs, arms or head.

The term “heat reflective inner surface” as used herein refers to the ability of the inner surface of the garment of the present invention to reflect heat inward towards the patient. The “heat reflective inner surface” will be more efficient at reflecting heat than traditional cotton or nylon. A “heat reflective inner surface” may be constructed by applying a heat reflective material, such as a metal or metal alloy, to the inner surface of the garment.

The term “medical access aperture” as used herein refers to a region that allows access to the patient suffering from or at risk of developing hypothermia. The “medical access aperture” may be a reversibly open and closeable aperture or slit. The “medical access aperture” may be positioned along a torso portion or a limb portion of the garment.

The term “passive heating” as used herein refers to warming a victim's body without applying an external heat source. “Passive heating” may occur from placing an individual or at least the torso of an individual in a closed environment such that the individual's own emitted body heat warms the environment. The efficiency of “passive heating” is increased when placing the individual's torso in a garment with a heat reflective inner surface.

The term “active heating” or “to actively heat” as used herein refers to warming a victim's body by applying a heat source external to the victim. “Active heating” may occur by positioning a dry active heating means to the garment of the present invention or may occur upon activation of a dry active heating means integrated to or positioned within the garment of the present invention.

The term “dry active heating means” as used herein refers to a device capable of releasing heat without exposing the patient or individual to an aqueous solution. A “dry active heating means” permits the patient to be warmed without adding significant moisture to the patient. A “dry active heating means” may be activated by initiating a chemical reaction within a closed container and the like. “Activating a dry active heating means” refers to the turning on or initiating a “dry active heating means,” which may be vary depending on the dry active heating means used, such that heat is released.

Garment

The present invention prevents or treats hypothermia by providing a garment that warms the torso at a greater rate than the extremities while providing regions of medical access to the individual. The greater warming rate of the torso facilitates blood flow away from the torso and towards the extremities thereby reducing the risk of heart attack during hypothermia medical treatment or examination. The disclosed garment may be used in the field such as by campers, survivalists, military personnel, ski patrol, and the like where treatment of hypothermia is common. However, the garment may also be used in conventional treatment facilities such as but not limited to hospitals, medical clinics, and the like. As will be envisioned, the features of the present invention allow an individual to receive a variety of medical treatments or examinations without substantially removing the garment.

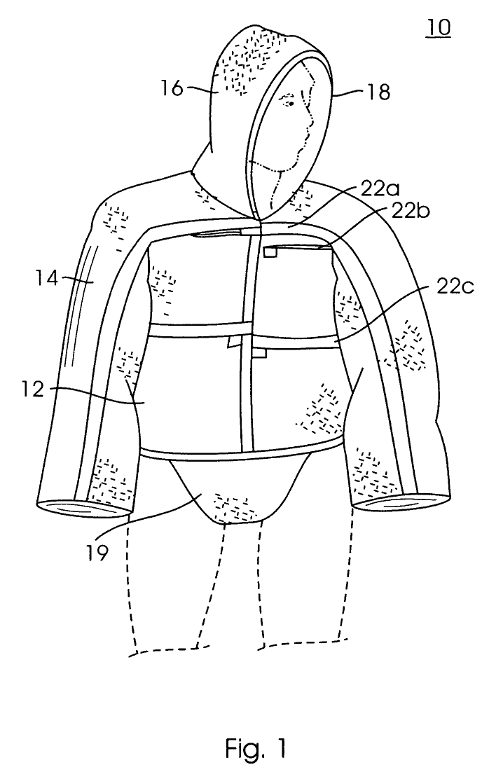

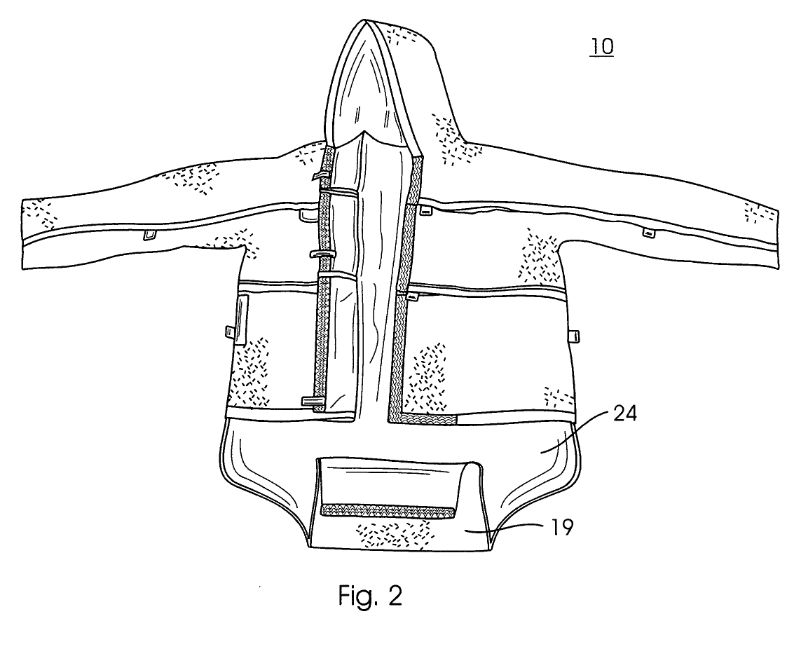

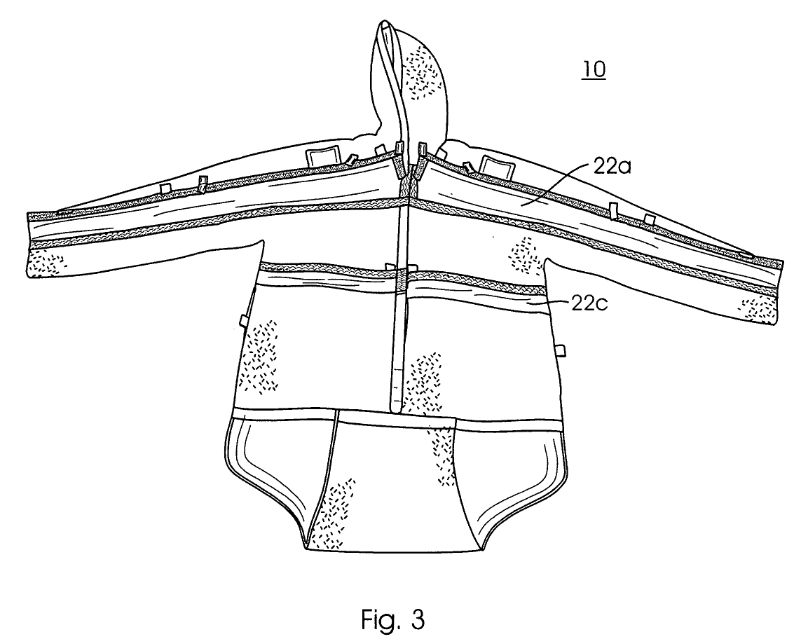

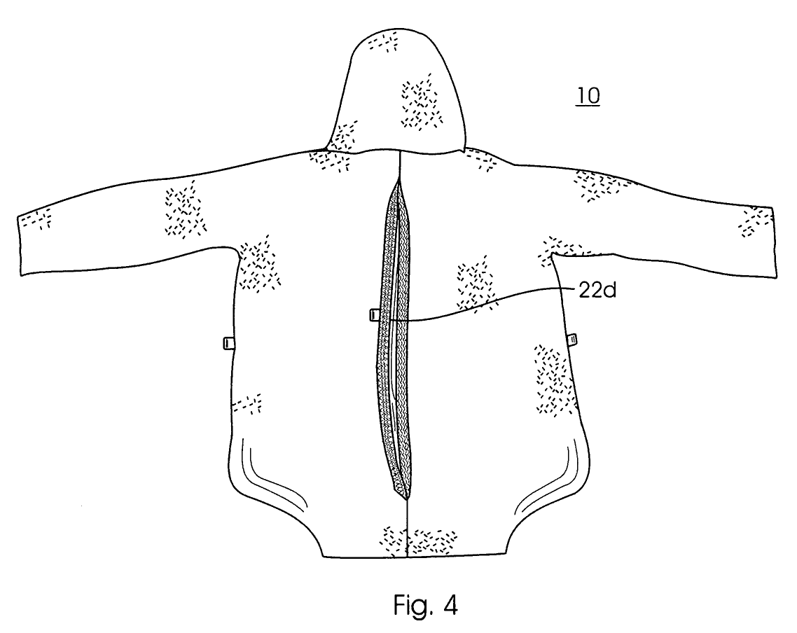

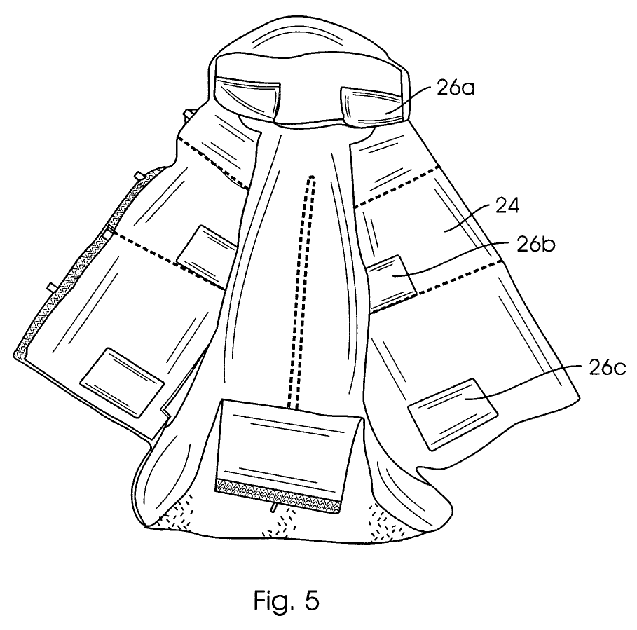

Referring to FIGS. 1-5, in one embodiment the present invention includes a hypothermia protection garment 10 including a reversibly closable upper body portion for substantially covering the upper body of an individual, the upper body portion including a torso portion 12 capable of substantially covering the torso of the individual, two sleeve portions 14 capable of substantially covering the arms and optionally the hands of the individual and a hood portion 16 capable of substantially covering the head of an individual, the hood portion 16 includes a face aperture or opening 18 for exposing a portion of the face of the individual. The upper body portion may also include a crotch flap 19 capable of substantially covering the crotch of the individual. The upper body portion has a heat reflective inner surface 24 and a heat positioning structure 26 for positioning a dry active heating means. The garment may also include one or more medical access apertures 22 capable of exposing a region of the individual for the application of a medical device or a medical procedure without substantial removal of the garment 10. The medical access aperture 22 may be reversibly opened and closed. The garment 10 may also include a lower portion including a single leg portion for two legs, two distinct leg portions for two legs and sock portions for feet. The lower portion and upper portion may be reversibly affixable or permanently affixed together. The garment may also include an integrated dry heating means or reversibly positionable dry heating means.

The hypothermia protection garment 10 is worn generally about the body and may reversibly open and close using a variety of techniques known in the garment industry such as but not limited to utilizing a zipper, button, hook and loop, snap, VELCRO and the like. The hypothermia protection garment 10 may include reversibly closable or adjustable ends generally corresponding to regions surrounding the face such as the face aperture 18, or where the head, neck, feet, legs, hands, wrists or arms exit the garment 10. Constructing an adjustable end may be performed by sewing a drawstring, elastic band or VELCRO closure to the garment 10. The closeable or adjustable end may further facilitate passive warming by preventing escape of the reflecting heat. There may also be adjustable regions throughout the garment such as an elastic or drawstring waist for better fit.

The hypothermia protection garment 10 should be sufficiently strong to prevent or reduce inadvertent tears or punctures and may be reinforced about the medical access aperture 22. The reinforcement may include additional material or increased stitching density. The garment 10 may be constructed from or coated with a variety of materials or fabrics known in the clothing, camping or medical device arts such as cotton, polyester, nylon, GORTEX and the like. The garment 10 may be constructed from polyethylene or laminated polyethylene. The garment 10 may be constructed from the same materials used in the construction of SPACE blankets (MPI Outdoors, Windham, N.H.). Preferably the garment 10 is constructed from a material or fabric that is lightweight, water resistant or water resistant treated and facilitates compact storage of the garment 10 when not in use such as polyethylene or laminated polyethylene.

The heat reflective inner surface 24 facilitates passive warming of an individual at risk of or suffering from hypothermia by substantially reflecting naturally emitted body heat back towards the body. Therefore heat reflective inner surface 24 may be constructed from a variety of fabrics or coatings that are substantially heat reflective. For example, metal or metal alloy coatings such as but not limited to aluminum, silver or gold coatings efficiently reflect heat and can be applied to a garment material such as polyethylene using vacuum depositing or laminating techniques. The coating may be applied using any known method such as spraying, laminating, gluing, sewing and the like. Preferably the heat reflective inner surface 24 is sufficiently malleable or bendable such that the garment 10 may be folded or bended without extensive difficulty. In one preferred embodiment the heat reflective inner surface 24 is constructed from the same material as the SPACE blanket (MPI Outdoor, Windham, N.H.).

The garment 10 may be formed using a variety of techniques known in the garment industry such as selecting the appropriate materials and constructing an appropriate size garment with desired features. The garment 10 may be constructed using techniques such as cutting aluminum laminated polyethylene sheets or sheets of SPACE blanket to an appropriate size or configuration, attaching a dry heating means positioning structure 26 to the cut sheets and attaching the cut sheets together to form a hypothermia protection suit. Attachment of materials used in the construction of the garment 10 may depend on the actual materials used. As guidance, materials may be attached by sewing, gluing, snapping, buttoning, zipping and the like. The garment 10 may be available in different sizes such as but not limited to small, medium, large, adult size, child size or may be one size fits all. The garment 10 may include an elastic portion or incorporate a drawstring for further size adjustment. One or more regions such as a hood 16, sleeves 14 or pant legs may be constructed such that the region is irreversibly or reversibly attached to the torso portion 12.

The hypothermia protection garment 10 may include one or more medical access apertures 22 able to accept a medical device or medical procedure. The medical access aperture 22 may be positioned at any suitable location on the upper portion or lower portion of the garment. Examples include but are not limited to the torso portion 12, the hood portion 16 or sleeve portion 14. The medical access aperture 22 permits examination of vital signs and the like or further medical treatment such as but not limited to placement of a stethoscope, intravenous (IV) lines or electrocardiogram (EKG) lines without the need for substantial removal of the garment 10. Preferably, the medical access aperture 22 is positioned generally about the regions of the garment where examination of an individual would more often occur. For example, a medical access aperture 22 positioned along the chest 22b may facilitate the attachment of electrodes for an EKG, or a stethoscope for examination of the heart or lungs. A medical access aperture positioned generally along the sleeves 22a may facilitate the placing of IV or examination of the individual's pulse. A medical access aperture along the hood may facilitate taking an individual's temperature or pulse along the neck region.

The hypothermia protection garment 10 includes a heat positioning structure 26 for positioning a dry heating means or includes an integrated dry heating means. Therefore, the dry active heating means may be permanently integrated such as sewn into the garment 10 or may be reversibly positionable such as inserting and removing the dry active heating means. In various embodiments, the heat positioning structure 26 may be provided in the configuration of a pocket, strap, snap, VELCRO and the like. In the preferred embodiment the heat postioning structure 26 is a pocket capapble of reversibly receiving a heating pad.

As indicated, the heat positioning structure 26 localizes or positions the dry heating means in a desired location. In one embodiment the heat positioning structure 26 positions the dry active heating means in close proximity to the victim's under arm 26b. In another embodiment, the heat positioning structure positions the dry active heating means in close proximity to the chest, crotch, hip region 26c or at the upper portion of the legs. In another embodiment, the positioning structure 26 positions the dry active heating means in close proximity to the victim's neck such as at the base of the hood 26a. A heat positioning structure 26 may also be located within a glove portion, a sock portion, and the like. Multiple heat positioning structures 26 may be located at multiple locations throughout the garment 10. The garment 10 may also include an integrated thermometer for monitoring the temperature within the garment 10 or the individual.

The dry active heating means may be any device able to release heat, that does not expose the individual to a foreign aqueous solution, is able to be placed within the hypothermia protection garment 10 and is able to be positioned using a positioning structure 26. For example, a variety of heat pads or warmers such as air-activated, chemical, and solid fuel heating pads are known in the survival and camping arts and can be used with the present invention. The dry active heating means is preferably portable. Companies such as Pristech (San Antonio, Tex.) and Boundtree (Dublin, Ohio) offer suitable warmers capable for use as a dry active heating means with the present invention. The active heating means may be a one-time use device or may be reusable. There may be one or more types of dry active heating means having the same or different heating capacities. Multiple dry active heating means may be utilized having temperature differences of 5 degrees, 7 degrees, 10 degrees Fahrenheit and the like. In these embodiment, a dry heating means with a higher temperature may be placed in closer proximity to the victim's heart than one having a lower temperature.

The present invention also includes methods of treating or preventing hypothermia using the disclosed garment 10. An individual at risk of developing or suffering from hypothermia is placed in a hypothermia protection garment 10 as substantially described. The garment 10 may be substantially closed around the torso, legs, arms, and head. The body is passively warmed by the reflection of the body's naturally emitted heat and optionally warmed by administering the active heating means. Medical evaluation or treatment is provided through a medical access aperture 22 without requiring substantial removal of the garment 10. In addition, the individual may be transported to another location without removal of the disclosed garments 10.

All headings are for the convenience of the reader and should not be used to limit the meaning of the text that follows the heading, unless so specified.

I claim:

1. A hypothermia protection garment comprising:
a reversibly closable upper body portion for substantially covering the upper body of an individual, said upper body portion including a torso portion capable of substantially covering the torso of the individual, two sleeve portions capable of substantially covering the arms and optionally the hands of the individual and a hood portion capable of substantially covering the head of an individual, said hood portion comprising a face aperture for exposing a portion of the face of the individual; and
said upper body portion having a heat reflective inner surface and a heat positioning structure for positioning a dry active heating means.

2. The hypothermia protection garment according to claim 1, wherein said upper body portion is water resistant.

3. The hypothermia protection garment according to claim 1, wherein said upper body portion is constructed from a material selected from the group consisting of cotton, nylon, polyester, GORTEX, polyethylene and laminated polyethylene.

4. The hypothermia protection garment according to claim 1, wherein said upper body portion is substantially tear resistant or substantially puncture resistant.

5. The hypothermia protection garment according to claim 1, wherein said torso portion further comprises a crotch flap capable of substantially covering the crotch of the individual, said crotch flap optionally including an additional heat positioning structure.

6. The hypothermia protection garment according to claim 1, wherein said heat reflective inner surface comprises a metal or metal alloy coating.

7. The hypothermia protection garment according to claim 1, wherein said heat reflective inner surface is selected from the group consisting of an aluminum coating, a silver coating and a gold coating.

8. The hypothermia protection garment according to claim 1, wherein said heat positioning structure is selected from the group consisting of a pocket, a strap, a snap and VELCRO.

9. The hypothermia protection garment according to claim 1, wherein said heat positioning structure positions said dry active heating means along said heat reflective inner surface at a region selected from the group consisting of an armpit region, a neck region, a hip region or a groin region.

10. The hypothermia protection garment according to claim 1, wherein said dry active heating means is a reusable heating pad or a one time use heating pad.

11. The hypothermia protection garment according to claim 1, wherein said upper portion further comprises a medical access aperture capable of exposing a region of the individual for performing a medical procedure or applying a medical device without substantial removal of the hypothermia protection garment.

12. The hypothermia protection garment according to claim 11, wherein said medical access aperture is a reversibly closable aperture.

13. The hypothermia protection garment according to claim 12, wherein said medical access aperture is positioned along a region selected from the group consisting of a chest region, an arm region and a back region.

14. The hypothermia protection garment according to claim 11, wherein said medical device is selected from the group consisting of an intravenous line, an electrocardiogram lead, a stethoscope, and a thermometer.

15. The hypothermia protection garment according to claim 1, further comprising a dry active heating means.

16. The hypothermia protection garment according to claim 15, wherein said dry active heating means is selected from the group consisting of a an air-activated heating pad, a chemical activated heating pad, a solid fuel heating pad.

17. The hypothermia protection garment according to claim 15, wherein said torso portion is heated at a greater rate than said sleeve portion.

18. The hypothermia protection garment according to claim 1, further comprising at a lower portion comprising two leg portions for substantially covering the legs of individual and optionally two feet portions for substantially covering the feet of an individual, wherein said lower body portion comprises a heat reflective inner surface.

19. A method of treating an individual at risk of developing or suffering from hypothermia comprising:
a) placing the individual in the hypothermia protection garment according to claim 1;
b) positioning a dry active heating means near a region selected from the group consisting of an armpit region, a neck region, a hip region or a groin region; and
c) activating said dry active heating means.

20. The method according to claim 19, further comprising monitoring the temperature of the individual and warming the individual's torso at a greater rate than the individual's arms or legs.

21. A method of treating an individual at risk of developing or suffering from hypothermia comprising:
a) placing the individual in the hypothermia protection garment according to claim 11;
b) positioning a dry active heating means near a region selected from the group consisting of an armpit region, a neck region, a hip region or a groin region; and
c) activating said dry active heating means; and
d) performing a medical procedure on the individual through said medical aperture.
